(19) United States
(12) Patent Application Publication
(10) Pub. No.: US 2022/0046954 A1
(43) Pub. Date: Feb. 17, 2022

(54) ANIMAL FEED COMPOSITION AND USE THEREOF

(71) Applicants: DSM IP ASSETS B.V., Heerlen (NL); NOVOZYMES A/S, Bagsvaerd (DK)

(72) Inventors: Raffaella AURELI, Kaiseraugst (CH); Leticia CARDOSO BITTENCOURT, Kaiseraugst (CH); Rual LOPEZ-ULIBARRI, Kaiseraugst (CH); Estefania PEREZ CALVO, Kaiseraugst (CH)

(21) Appl. No.: 17/274,619

(22) PCT Filed: Sep. 11, 2019
(86) PCT No.: PCT/EP2019/074221

§ 371 Date: Mar. 9, 2021

(30) Foreign Application Priority Data
Sep. 11, 2018 (EP) 18193727.7

(51) Int. Cl. A23K 20/189; A23K 20/174; A23K 20/142; A23K 20/20; A23K 50/75; A23K 10/30

(52) U.S. Cl. CPC A23K 20/189; A23K 20/174; A23K 10/30; A23K 20/20; A23K 50/75; A23K 20/142

(57) ABSTRACT

The present invention relates to a method of improving flock uniformity and/or meat quality of a monogastric animal comprising administering to the animal a composition, an animal feed or an animal feed additive comprising one or more microbial muramidases.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to methods of improving flock uniformity and/or meat quality of an animal using one or more microbial muramidase.

### Description of the Related Art

Muramidase, also named as lysozyme, is an O-glycosyl hydrolase produced as a defensive mechanism against bacteria by many organisms. The enzyme causes the hydrolysis of bacterial cell walls by cleaving the glycosidic bonds of peptidoglycan, an important structural molecule in bacteria. After having their cell walls weakened by muramidase action, bacterial cells lyse as a result of unbalanced osmotic pressure.

Muramidase naturally occurs in many organisms such as viruses, plants, insects, birds, reptiles and mammals. Muramidase has been classified into five different glycoside hydrolase (GH) families (CAZy, www.cazy.org): hen egg-white muramidase (GH22), goose egg-white muramidase (GH23), bacteriophage T4 muramidase (GH24), Sphingomonas flagellar protein (GH73) and Chalaropsis muramidases (GH25). Muramidases from the families GH23 and GH24 are primarily known from bacteriophages and have only recently been identified in fungi. The muramidase family GH25 has been found to be structurally unrelated to the other muramidase families.

Muramidase has traditionally been extracted from hen egg white due to its natural abundance and until very recently hen egg white muramidase was the only muramidase investigated for use in animal feed. Muramidase extracted from hen egg white is the primary product available on the commercial market, but does not cleave N,6-O-diacetylmuramic acid in e.g. Staphylococcus aureus cell walls and is thus unable to lyse this important human pathogen among others (Masschalck B, Deckers D, Michiels C W (2002), “Lytic and nonlytic mechanism of inactivation of gram-positive bacteria by muramidase under atmospheric and high hydrostatic pressure”, J Food Prot. 65(12):1916-23).

WO2000/21381 discloses a composition comprising at least two antimicrobial enzymes and a polyunsaturated fatty acid, wherein one of the antimicrobial enzymes was a GH22 muramidase from chicken egg white. GB2379166 discloses a composition comprising a compound that disrupts the peptidoglycan layer of bacteria and a compound that disrupts the phospholipid layer of bacteria, wherein the peptidoglycan disrupting compound was a GH22 muramidase from chicken egg white.

WO2004/026334 discloses an antimicrobial composition for suppressing the growth of enteric pathogens in the gut of livestock comprising (a) a cell wall lysing substance or its salt, (b) a antimicrobial substance, (c) a sequestering agent and (d) a lantibiotic, wherein the cell wall lysing substance or its salt is a GH22 muramidase from hen egg white.

Surprisingly, the inventors of the present invention discovered that muramidases can be used in feed to improve flock uniformity and/or meat quality of a monogastric animal. As demand on animal protein is growing, such solution which improves growth performance of an animal is always of interest of farmers.

### SUMMARY OF THE INVENTION

Accordingly, the present invention provides a method for improving flock uniformity and/or meat quality of a monogastric animal comprising administering to the animal a composition, an animal feed or an animal fee additive comprising one or more microbial muramidases.

### Overview of Sequence Listing

SEQ ID NO: 1 is the mature amino acid sequence of a wild type GH25 muramidase from Acremonium alcalophilum with N-terminal SPIRR as described in WO 2013/076253.

SEQ ID NO: 2 is the gene sequence of the GH24 muramidase as isolated from Trichophaea saccata.

SEQ ID NO: 3 is the amino acid sequence as deduced from SEQ ID NO: 2.

SEQ ID NO: 4 is the mature amino acid sequence of a wild type GH24 muramidase from Trichophaea saccata.

SEQ ID NO: 5 is the mature amino acid sequence of a wild type GH22 muramidase from Gallus gallus (hen egg white muramidase).

SEQ ID NO: 6 is primer F-80470.

SEQ ID NO: 7 is primer R-80470.

SEQ ID NO: 8 is primer 8643.

SEQ ID NO: 9 is primer 8654.

SEQ ID NO: 10 is the mature amino acid sequence of a wild type GH25 muramidase from Acremonium alcalophilum as described in WO 2013/076253.

### Definitions

Microbial muramidase: The term “microbial muramidase” means a polypeptide having muramidase activity which is obtained or obtainable from a microbial source. Examples of microbial sources are fungi; i.e. the muramidase is obtained or obtainable from the kingdom Fungi, wherein the term kingdom is the taxonomic rank. In particular, the the microbial muramidase is obtained or obtainable from the phylum Ascomycota, such as the sub-phylum Pezizomycotina, wherein the terms phylum and sub-phylum is the taxonomic ranks.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Muramidase activity: The term “muramidase activity” means the enzymatic hydrolysis of the 1,4-beta-linkages between N-acetylmuramic acid and N-acetyl-D-glucosamine residues in a peptidoglycan or between N-acetyl-D-glucosamine residues in chitodextrins, resulting in bacteriolysis due to osmotic pressure. Muramidase belongs to the enzyme class EC 3.2.1.17. Muramidase activity is typically measured by turbidimetric determination. The method is based on the changes in turbidity of a suspension of Micrococcus luteus ATCC 4698 induced by the lytic action of muramidase. In appropriate experimental conditions these changes are proportional to the amount of muramidase in the medium (c.f. INS 1105 of the Combined Compendium of Food Additive Specifications of the Food and Agriculture Organisation of the UN (www.fao.org)). For the purpose of the present invention, muramidase activity is determined according to the turbidity assay described in example 5 (“Determination of Muramidase Activity”). In one aspect, the polypeptides of the present invention have at least 20%, e.g., at least 40%, at least 50%, at least 60%, at least 70%, at least 80%, at least 90%, at least 95%, or at least 100% of the muramidase activity of SEQ ID NO: 1. In one aspect, the polypeptides of the present invention have at least 20%, e.g., at least 40%, at least 50%, at least 60%, at least 70%, at least 80%, at least 90%, at least 95%, or at least 100% of the muramidase activity of SEQ ID NO: 4. In one aspect, the polypeptides of the present invention have at least 20%, e.g., at least 40%, at least 50%, at least 60%, at least 70%, at least 80%, at least 90%, at least 95%, or at least 100% of the muramidase activity of SEQ ID NO: 10.

Fragment: The term “fragment” means a polypeptide or a catalytic domain having one or more (e.g., several) amino acids absent from the amino and/or carboxyl terminus of a mature polypeptide or domain; wherein the fragment has muramidase activity. In one aspect, a fragment comprises at least 170 amino acids, such as at least 175 amino acids, at least 177 amino acids, at least 180 amino acids, at least 185 amino acids, at least 190 amino acids, at least 195 amino acids or at least 200 amino acids of SEQ ID NO: 1 and has muramidase activity.

In another aspect, a fragment comprises at least 210 amino acids, such as at least 215 amino acids, at least 220 amino acids, at least 225 amino acids, at least 230 amino acids, at least 235 amino acids or at least 240 amino acids of SEQ ID NO: 4 and has muramidase activity.

In one aspect, a fragment comprises at least 170 amino acids, such as at least 175 amino acids, at least 177 amino acids, at least 180 amino acids, at least 185 amino acids, at least 190 amino acids, at least 195 amino acids or at least 200 amino acids of SEQ ID NO: 10 and has muramidase activity.

Isolated: The term “isolated” means a substance in a form that environment does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

Variant: The term “variant” means a polypeptide having muramidase activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a muramidase variant according to the invention may comprise from 1 to 5; from 1 to 10; from 1 to 15; from 1 to 20; from 1 to 25; from 1 to 30; from 1 to 35; from 1 to 40; from 1 to 45; or from 1-50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 alterations and have at least 20%, e.g., at least 40%, at least 50%, at least 60%, at least 70%, at least 80%, at least 90%, at least 95%, or at least 100% of the muramidase activity of the parent muramidase, such as SEQ ID NO: 1, SEQ ID NO: 4 or SEQ ID NO: 10.

Monogastric animal: The term “monogastric animal” refers to any animal which has a simple single-chambered stomach except humans. Examples of monogastric animals include pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer, hens (referred to herein as layers)); pet animals such as cat and dog; horses (including but not limited to hotbloods, coldbloods and warm bloods), crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Roughage: The term “roughage” means dry plant material with high levels of fiber, such as fiber, bran, husks from seeds and grains and crop residues (such as stover, copra, straw, chaff, sugar beet waste).

Flock uniformity: The term “flock uniformity” means the uniformity of body weights within the flock of animals. Uniformity is expressed as the percent of individual weights which occur within 10% of the current flock average. Flocks lacking uniformity are more difficult to design diets for (because there is a mixture of light and heavy animals in the flock).

Meat quality: The term “meat quality” is defined as a measurement of attributes or characters that determine the suitability of meat to be eaten as fresh or stored for reasonable period without deterioration. Many factors affect the fat, lean and connective tissue component of meat and therefore influence meat quality. Meat quality may be characterized by texture, colour, juiciness, and/or yield of eatable parts (such as carcass) or favourable parts (such as breast filet of chicken), etc.

### DETAILED DESCRIPTION OF THE INVENTION

Methods of Improving Flock Uniformity and/or Meat Quality

It has been surprisingly found that supplementing an animal feed with a microbial muramidase results in a significant benefit of improving flock uniformity of a monogastric animal, compared to an animal feed without the microbial muramidase.

It has been further surprisingly found that supplementing an animal feed with a microbial muramidase results in improving meat quality of a monogastric animal, compared to an animal feed without the microbial muramidase. In in vivo broiler trials, it was surprisingly discovered that:
(a) treatment with a muramidase leads to a higher yield of breast meat of the broilers;
(b) treatment with a muramidase leads to a higher yield of breast filet of the broilers; and/or
(c) treatment with a muramidase leads to a higher yield of carcass of the broilers.

Thus the invention relates to a method of improving flock uniformity and/or meat quality of a monogastric animal comprising administering to the animal a composition, an animal feed or an animal feed additive comprising one or more microbial muramidases.

In the present invention, the improvement is compared to an animal feed or animal feed additive wherein the microbial muramidase is not present (herein referred to as the negative control).

Preferably, the flock uniformity is increased by at least 1%, such as by at least 1.5%, at least 2.0%, at least 2.5%, at least 3%, at least 3.5%, at least 4% or at least 5% compared to the negative control.

Preferably, the meat quality is increased by at least 10%, such as by at least 15%, at least 25%, or at least 30% compared to the negative control.

In the present invention, the microbial muramidase may be dosed at a level of 100 to 1000 mg enzyme protein per kg animal feed, such as 200 to 900 mg, 300 to 800 mg, 400 to 700 mg, 500 to 600 mg enzyme protein per kg animal feed, or any combination of these intervals.

In the present invention, the monogastric animal may be selected from the group consisting of swine, piglet, growing pig, sow, poultry, turkey, duck, quail, guinea fowl, goose, pigeon, squab, chicken, broiler, layer, pullet and chick, cat, dog, horse, crustaceans, shrimps, prawns, fish, amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish. Preferably, the monogastric animal is a selected from the group consisting of swine, piglet, growing pig, sow, poultry, turkey, duck, quail, guinea fowl, goose, pigeon, squab, chicken, broiler, layer, pullet and chick. More preferably, the the monogastric animal is a selected from the group consisting of swine, piglet, growing pig, sow, chicken, broiler, layer, and chick.

In the present invention, the microbial muramidase may be fed to the animal from birth until slaughter. Preferably, the the microbial muramidase is fed to the animal on a daily basis from birth until slaughter. More Preferably, the microbial muramidase is fed to the animal on a daily basis for at least 10 days, such as at least 15 days or at least 20 days (where the days can be continuous or non-continuous) during the life span of the animal. Further preferably, the microbial muramidase is fed to the animal for 10-20 days followed by a non-treatment period of 5-10 days, and this cycle is repeated during the life span of the animal.

In the present invention, the microbial muramidase may be fed to broilers for the first 49 days after hatching. Preferably, the microbial muramidase is fed to broilers for the first 36 days after hatching. More preferably, the microbial muramidase is fed to broilers on days 22 to 36 after hatching. Further preferably, the microbial muramidase is fed to broilers during the pre-starter (days 1-7) period. Further preferably, the microbial muramidase is fed to broilers during the starter (days 8-22) period. Further preferably, the microbial muramidase is fed to broilers during the pre-starter (days 1-7) and starter (days 8-22) period.

In the present invention, the microbial muramidase may be fed to layers during the life span of the animal. Preferably, the microbial muramidase is fed to layers for 76 weeks from hatching. More preferably, the microbial muramidase is fed to layers during the laying period, (from ca. week 18). Further preferably, the microbial muramidase is fed to layers during the laying period but withheld during the forced molting period.

In the present invention, the microbial muramidase may be fed to turkeys during life span of the animal. Preferably, the microbial muramidase is fed to turkeys for 24 weeks from hatching. More preferably, the microbial muramidase is fed to turkeys for the first 16 weeks from hatching (for hens) and for the first 20 weeks for hatching (for toms).

In the present invention, the microbial muramidase may be fed to swine during life span of the animal. Preferably, the microbial muramidase is fed to swine for 27 weeks from birth. More preferably, the microbial muramidase is fed to piglets from birth to weaning (at 4 weeks). Further preferably, the microbial muramidase is fed to piglets for the first 6 weeks from birth (4 weeks of lactation and 2 weeks post-weaning). Further preferably, the microbial muramidase is fed to weaning piglets during the pre-starter (days 1-14 after weaning). Further preferably, the microbial muramidase is fed to weaning piglets during the starter (days 15-42 after weaning) period. Further preferably, the microbial muramidase is fed to weaning piglets during the pre-starter (days 1-14 after weaning) and starter (days 15-42 after weaning) period. Further preferably, the microbial muramidase is fed to swine during the grower/fattening period (week 10 to ca. week 27 after birth).

In the present invention, the microbial muramidase may be of fungal origin. Preferably, the microbial muramidase is obtained or obtainable from the phylum Ascomycota, such as the sub-phylum Pezizomycotina. Preferably, the microbial muramidase comprises one or more domains selected from the list consisting of GH24 and GH25.

In the present invention, the microbial muramidase may have at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1, 4 or 10.

In the present invention, the microbial muramidase may comprise or consist of the amino acid sequence of SEQ ID NO: 1 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. Preferably, the microbial muramidase comprises or consists of the amino acid sequence of SEQ ID NO: 1 or an allelic variant thereof and a N-terminal and/or C-terminal His-tag and/or HQ-tag. More preferably, the polypeptide comprises or consists of amino acids 1 to 213 of SEQ ID NO: 1.

Alternatively, the microbial muramidase may comprise or consist of the amino acid sequence of SEQ ID NO: 4 or an allelic variant thereof; or is a fragment thereof having muramidase activity, wherein the fragment comprises at least 210 amino acids, such as at least 215 amino acids, at least 220 amino acids, at least 225 amino acids, at least 230 amino acids, at least 235 amino acids or at least 240 amino acids. Preferably, the microbial muramidase comprises or consists of the amino acid sequence of SEQ ID NO: 4 or an allelic variant thereof and a N-terminal and/or C-terminal His-tag and/or HQ-tag. More preferably, the polypeptide comprises or consists of amino acids 1 to 245 of SEQ ID NO: 4.

More alternatively, the microbial muramidase may comprise or consist of the amino acid sequence of SEQ ID NO: 10 or an allelic variant thereof; or is a fragment thereof having muramidase activity, wherein the fragment comprises at least 210 amino acids, such as at least 215 amino acids, at least 220 amino acids, at least 225 amino acids, at least 230 amino acids, at least 235 amino acids or at least 240 amino acids. Preferably, the microbial muramidase comprises or consists of the amino acid sequence of SEQ ID NO: 10 or an allelic variant thereof and a N-terminal and/or C-terminal His-tag and/or HQ-tag. More preferably, the polypeptide comprises or consists of amino acids 1 to 208 of SEQ ID NO: 10.

In the present invention, the microbial muramidase may be a variant of SEQ ID NO: 1, 4 or 10 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. Preferably, the number of positions comprising one or more amino acid substitutions, and/or one or more amino acid deletions, and/or one or more amino acid insertions or any combination thereof in SEQ ID NO: 1, 4 or 10 is between 1 and 45, such as 1-40, 1-35, 1-30, 1-25, 1-20, 1-15, 1-10 or 1-5 positions. More preferably, the number of positions comprising one or more amino acid substitutions, and/or one or more amino acid deletions, and/or one or more amino acid insertions or any combination thereof in SEQ ID NO: 1, 4 or 10 is not more than 10, e.g., 1, 2, 3, 4, 5, 6, 7, 8, 9 or 10. Further preferably, the number of substitutions, deletions, and/or insertions in SEQ ID NO: 1, 4 or 10 is not more than 10, e.g., 1, 2, 3, 4, 5, 6, 7, 8, 9 or 10. Further preferably, the number of substitutions, preferably conservative substitutions, in SEQ ID NO: 1, 4 or 10 is not more than 10, e.g., 1, 2, 3, 4, 5, 6, 7, 8, 9 or 10. Further preferably, the number of conservative substitutions in SEQ ID NO: 1, 4 or 10 is not more than 10, e.g., 1, 2, 3, 4, 5, 6, 7, 8, 9 or 10.

Any person skilled in the art can understand, the polypeptide of the microbial muramidase may have amino acid changes. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for muramidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The crystal structure of the Acremonium alcalophilum CBS114.92 muramidase was solved at a resolution of 1.3 Å as disclosed in WO 2013/076253. These atomic coordinates can be used to generate a three dimensional model depicting the structure of the Acremonium alcalophilum CBS114.92 muramidase or homologous structures (such as the variants of the present invention). Using the x/ray structure, amino acid residues D95 and E97 (using SEQ ID NO: 1 for numbering) were identified as catalytic residues.

In one embodiment, the invention relates to a method of improving flock uniformity and/or meat quality of a monogastric animal comprising administering to the animal a composition, an animal feed or an animal feed additive comprising one or more microbial muramidases, wherein:

(a) the microbial muramidase is a microbial muramidase comprising one or more domains selected from the list consisting of GH24 and GH25, is dosed at a level of 300 to 500 mg enzyme protein per kg animal feed;

(b) the animal is a selected from the group consisting of swine, piglet, growing pig, sow, chicken, broiler, layer, pullet and chick;

(c) optionally the microbial muramidase is fed to the animal on a daily basis for at least 10 days during the life span of the animal.

In another embodiment, the invention relates to a method of improving flock uniformity and/or meat quality of a monogastric animal comprising administering to the animal a composition, an animal feed or an animal feed additive comprising one or more microbial muramidases, wherein:

(a) the microbial muramidase is a GH24 or GH 25 muramidase obtained or obtainable from the phylum Ascomycota, and is dosed at a level of 300 to 500 mg enzyme protein per kg animal feed;

(b) the animal is a selected from the group consisting of swine, piglet, growing pig, sow, chicken, broiler, layer, pullet and chick; and

(c) the flock uniformity is increased by at least 1% compared to the negative control.

In another embodiment, the invention relates to a method of improving flock uniformity and/or meat quality of a monogastric animal comprising administering to the animal a composition, an animal feed or an animal feed additive comprising one or more microbial muramidases, wherein:

(a) the microbial muramidase is a GH24 or GH25 muramidase obtained or obtainable from the phylum Ascomycota, is dosed at a level of 300 to 500 mg enzyme protein per kg animal feed;

(b) the animal is a selected from the group consisting of swine, piglet, growing pig, sow, chicken, broiler, layer, pullet and chick; and

(c) the meat quality is increased by at least 10% compared to the negative control.

### Formulation

The microbial muramidase of the present invention may be formulated as a composition for improving flock uniformity and/or meat quality of a monogastric animal, which is also the present invention intents to cover. The microbial muramidase of the present invention may be formulated as a liquid or a solid.

For a liquid formulation, the formulating agent may comprise a polyol (such as e.g. glycerol, ethylene glycol or propylene glycol), a salt (such as e.g. sodium chloride, sodium benzoate, potassium sorbate) or a sugar or sugar derivative (such as e.g. dextrin, glucose, sucrose, and sorbitol). Thus the composition of the present invention may a liquid composition comprising the microbial muramidase of the present invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the animals.

For a solid formulation, the composition of the present invention may be for example as a granule, spray dried powder or agglomerate. The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

For example, the solid composition is in granulated form. The granule may have a matrix structure where the components are mixed homogeneously. However, the granule typically comprises a core particle and one or more coatings, which typically are salt and/or wax coatings. Examples of waxes are polyethylene glycols; polypropylenes; Carnauba wax; Candelilla wax; bees wax; hydrogenated plant oil or animal tallow such as hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil. The core particle can either be a homogeneous blend of muramidase of the invention optionally combined with one or more additional enzymes and optionally together with one or more salts or an inert particle with the muramidase of the invention optionally combined with one or more additional enzymes applied onto it.

In the above granule, the material of the core particles may be selected from the group consisting of inorganic salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals and clay minerals (also known as hydrous aluminium phyllosilicates). Preferably, the core comprises a clay mineral such as kaolinite or kaolin.

The salt coating is typically at least 1 μm thick and can either be one particular salt or a mixture of salts, such as Na₂SO₄, K₂SO₄, MgSO₄ and/or sodium citrate. Other examples are those described in e.g. WO 2008/017659, WO 2006/034710, WO 1997/05245, WO 1998/54980, WO 1998/55599, WO 2000/70034 or polymer coating such as described in WO 2001/00042.

Preferably, the composition of the present invention is a solid composition comprising the muramidase of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. More preferably, the formulating agent is selected from one or more of the following compounds: sodium sulfate, dextrin, cellulose, sodium thiosulfate and calcium carbonate. Further preferably, the solid composition is in granulated form. More further preferably, the solid composition is in granulated form and comprises a core particle, an enzyme layer comprising the muramidase of the invention and a salt coating.

Preferably, the formulating agent is selected from one or more of the following compounds: glycerol, ethylene glycol, 1, 2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose. More preferably, the formulating agent is selected from one or more of the following compounds: 1, 2-propylene glycol, 1, 3-propylene glycol, sodium sulfate, dextrin, cellulose, sodium thiosulfate, kaolin and calcium carbonate.

### Animal Feed and Animal Feed Additives

The microbial muramidase of the present invention may also be formulated as animal feed or animal feed additive for improving flock uniformity and/or meat quality of an animal, which is also the present invention intents to cover.

Animal feed compositions or diets have a relatively high content of protein. Poultry and pig diets can be characterised as indicated in Table B of WO 2001/058275, columns 2-3. Fish diets can be characterised as indicated in column 4 of this Table B. Furthermore such fish diets usually have a crude fat content of 200-310 g/kg.

An animal feed composition according to the present invention may have a crude protein content of between 50 and 800 g/kg, and furthermore comprises one or more microbial muramidases as described herein.

Furthermore, or in the alternative (to the crude protein content indicated above), the animal feed composition of the present invention may have a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

Particularly, the content of metabolisable energy, crude protein, calcium, phosphorus, methionine, methionine plus cysteine, and/or lysine may be within any one of ranges 2, 3, 4 or 5 in Table B of WO 2001/058275 (R. 2-5).

The nitrogen content is determined by the Kjeldahl method (A.O.A.C., 1984, Official Methods of Analysis 14th ed., Association of Official Analytical Chemists, Washington D.C.) and crude protein is calculated as nitrogen (N) multiplied by a factor 6.25 (i.e. Crude protein (g/kg)=N (g/kg)×6.25).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

The animal feed composition of the present invention may contain at least one vegetable protein as defined above.

The animal feed composition of the present invention may also contain animal protein, such as Meat and Bone Meal, Feather meal, and/or Fish Meal, typically in an amount of 0-25%. The animal feed composition of the present invention may also comprise Dried Distillers Grains with Solubles (DDGS), typically in amounts of 0-30%.

Preferably, the animal feed composition of the present invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

Preferably, the animal feed of the present invention comprises vegetable proteins. The protein content of the vegetable proteins is at least 10, 20, 30, 40, 50, 60, 70, 80, or 90% (w/w).

In the present invention, the vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

The vegetable protein source may be material from one or more plants of the family Fabaceae, e.g., soybean, lupine, pea, or bean. The vegetable protein source may also be material from one or more plants of the family Chenopodiaceae, e.g. beet, sugar beet, spinach or quinoa. Other examples of vegetable protein sources are rapeseed, and cabbage. Soybean is a preferred vegetable protein source. Other examples of vegetable protein sources are cereals such as barley, wheat, rye, oat, maize (corn), rice, and sorghum.

Animal diets can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) muramidase/enzyme preparation may also be added before or during the feed ingredient step. Typically a liquid enzyme preparation comprises the microbial muramidase of the present invention optionally with a polyol, such as glycerol, ethylene glycol or propylene glycol, and is added after the pelleting step, such as by spraying the liquid formulation onto the pellets. The muramidase may also be incorporated in a feed additive or premix.

Alternatively, the microbial muramidase of the present invention may be prepared by freezing a mixture of liquid enzyme solution with a bulking agent such as ground soybean meal, and then lyophilizing the mixture.

In the present invention, the animal feed composition may further comprise one or more additional enzymes, microbes, vitamins, minerals, amino acids, and/or other feed ingredients.

Preferably, the composition comprises one or more of the microbial muramidases of the present invention, one or more formulating agents and one or more components selected from the list consisting of: one or more additional enzymes; one or more microbes; one or more vitamins; one or more minerals; one or more amino acids; and one or more other feed ingredients.

The final muramidase concentration in the animal feed composition of the present invention may be within the range of 0.01-200 mg enzyme protein per kg animal feed, such as 0.1 to 150 mg, 0.5 to 100 mg, 1 to 75 mg, 2 to 50 mg, 3 to 25 mg, 2 to 80 mg, 5 to 60 mg, 8 to 40 mg or 10 to 30 mg enzyme protein per kg animal feed, or any combination of these intervals.

It is at present contemplated that the microbial muramidase is administered in one or more of the following amounts (dosage ranges): 0.01-200; 0.01-100; 0.5-100; 1-50; 5-100; 5-50; 10-100; 0.05-50; 5-25; or 0.10-10—all these ranges being in mg muramidase per kg feed (ppm).

For determining mg muramidase protein per kg feed, the muramidase is purified from the feed composition, and the specific activity of the purified muramidase is determined using a relevant assay (see under muramidase activity). The muramidase activity of the feed composition as such is also determined using the same assay, and on the basis of these two determinations, the dosage in mg muramidase protein per kg feed is calculated.

The animal feed additive of the present invention is intended for being included (or prescribed as having to be included) in animal diets or feed at levels of 0.01 to 10.0%; more particularly 0.05 to 5.0%; or 0.2 to 1.0% (% meaning g additive per 100 g feed). This is so in particular for premixes.

The same principles apply for determining mg muramidase protein in feed additives. Of course, if a sample is available of the muramidase used for preparing the feed additive or the feed, the specific activity is determined from this sample (no need to purify the muramidase from the feed composition or the additive).

### Additional Enzymes

In the present invention, the compositions or animal feed or animal feed additive described herein optionally include one or more enzymes. Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase, muramidase and galactosidase is described in Henrissat et al, “The carbohydrate-active enzymes database (CAZy) in 2013”, Nucl. Acids Res. (1 Jan. 2014) 42 (D1): D490-D495; see also www.cazy.org.

Thus the composition or animal feed or animal feed additive of the present invention may also comprise at least one other enzyme selected from the group consisting of phytase (EC 3.1.3.8 or 3.1.3.26), xylanase (EC 3.2.1.8); galactanase (EC 3.2.1.89); alpha-galactosidase (EC 3.2.1.22); protease (EC 3.4); phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); arabinofuranosidase (EC 3.2.1.55); beta-xylosidase (EC 3.2.1.37); acetyl xylan esterase (EC 3.1.1.72); feruloyl esterase (EC 3.1.1.73); cellulase (EC 3.2.1.4); cellobiohydrolases (EC 3.2.1.91); beta-glucosidase (EC 3.2.1.21); pullulanase (EC 3.2.1.41), alpha-mannosidase (EC 3.2.1.24), mannanase (EC 3.2.1.25) and beta-glucanase (EC 3.2.1.4 or EC 3.2.1.6), or any combination thereof.

Examples of commercially available phytases include Bio-Feed™ Phytase (Novozymes), Ronozyme® P, Ronozyme® NP and Ronozyme® HiPhos (DSM Nutritional Products), Natuphos™ (BASF), Finase® and Quantum® Blue (AB Enzymes), OptiPhos® (Huvepharma) Phyzyme® XP (Verenium/DuPont) and Axtra® PHY (DuPont). Other preferred phytases include those described in e.g. WO 98/28408, WO 00/43503, and WO 03/066847.

Examples of commercially available xylanases include Ronozyme® WX and Ronozyme® G2 (DSM Nutritional Products), Econase® XT and Barley (AB Vista), Xylathin® (Verenium), Hostazym® X (Huvepharma) and Axtra® XB (Xylanase/beta-glucanase, DuPont).

Examples of commercially available proteases include Ronozyme® ProAct (DSM Nutritional Products).

### Microbes

In the present invention, the composition or animal feed or animal feed additive may further comprise one or more additional microbes. For example, the composition or animal feed further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

Preferably, the composition or animal feed or animal feed additive of the present invention further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, Lactobacillus rhamnosus, Clostridium butyricum, Bifidobacterium animalis ssp. animalis, Lactobacillus reuteri, Lactobacillus salivarius ssp. salivarius, Megasphaera elsdenii, Propionibacteria sp.

More preferably, the composition or animal feed or animal feed additive of the present invention further comprises a bacterium from one or more of the following strains of Bacillus subtilis: 3A-P4 (PTA-6506), 15A-P4 (PTA-6507), 22C-P1 (PTA-6508), 2084 (NRRL B-500130), LSSA01 (NRRL-B-50104), BS27 (NRRL B-501 05), BS 18 (NRRL B-50633), BS 278 (NRRL B-50634), DSM 29870, DSM 29871, NRRL B-50136, NRRL B-50605, NRRL B-50606, NRRL B-50622 and PTA-7547.

More preferably, the composition, animal feed or animal feed additive of the present invention further comprises a bacterium from one or more of the following strains of Bacillus pumilus: NRRL B-50016, ATCC 700385, NRRL B-50885 or NRRL B-50886.

More preferably, composition, animal feed additive or animal feed further comprises a bacterium from one or more of the following strains of Bacillus lichenformis: NRRL B 50015, NRRL B-50621 or NRRL B-50623.

More preferably, the composition, animal feed or animal feed additive of the present invention further comprises a bacterium from one or more of the following strains of Bacillus amyloliquefaciens: DSM 29869, DSM 29872, NRRL B 50607, PTA-7543, PTA-7549, NRRL B-50349, NRRL B-50606, NRRL B-50013, NRRL B-50151, NRRL B-50141, NRRL B-50147 or NRRL B-50888.

The bacterial count of each of the bacterial strains in the composition, animal feed or animal feed additive of the present invention is between 1×10⁴ and 1×10¹⁴ CFU/kg of dry matter, preferably between 1×10⁶ and 1×10¹² CFU/kg of dry matter, more preferably between 1×10⁷ and 1×10¹¹, and the most preferably between 1×10⁸ and 1×10¹⁰ CFU/kg of dry matter.

The bacterial count of each of the bacterial strains in the composition, animal feed or animal feed additive of the present invention is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day, and the most preferably between 1×10⁹ and 1×10¹¹ CFU/animal/day.

In the present invention, the one or more bacterial strains may be present in the form of a stable spore.

### Premix

In the present invention, the composition, animal feed or animal feed additive may include a premix, comprising e.g. vitamins, minerals, enzymes, amino acids, preservatives, antibiotics, other feed ingredients or any combination thereof which are mixed into the animal feed.

### Amino Acids

the composition, animal feed or animal feed additive of the present invention may further comprise one or more amino acids. Examples of the amino acids include but are not limited to lysine, alanine, beta-alanine, threonine, methionine and tryptophan.

### Vitamins and Minerals

In the present invention, the composition, animal feed or animal feed additive may include one or more vitamins, such as one or more fat-soluble vitamins and/or one or more water-soluble vitamins. Optionally, the composition, animal feed or animal feed additive of the present invention may include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, selenium and zinc.

Non-limiting examples of macro minerals include calcium, magnesium, potassium and sodium.

The nutritional requirements of these components (exemplified with poultry and piglets/pigs) are listed in Table A of WO 2001/058275. Nutritional requirement means that these components should be provided in the diet in the concentrations indicated.

In the alternative, the composition, animal feed or animal feed additive of the present invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the composition, animal feed or animal feed additive of the present invention in such an amount as to provide an in-feed-concentration within the range indicated in column four, or column five, or column six of Table A.

Preferably, the animal feed additive of the invention comprises at least one of the below vitamins, to provide an in-feed-concentration within the ranges specified in the below Table 1 (for piglet and broiler diets, respectively).

TABLE 1
Typical vitamin recommendations

| Vitamin | Piglet diet | | Broiler diet | |
|---|---|---|---|---|
| Vitamin A | 10,000-15,000 | | 8-12,500 | IU/kg feed |
| | IU/kg feed | | | |
| Vitamin D3 | 1800-2000 | IU/kg feed | 3000-5000 | IU/kg feed |
| Vitamin E | 60-100 | mg/kg feed | 150-240 | mg/kg feed |
| Vitamin K3 | 2-4 | mg/kg feed | 2-4 | mg/kg feed |
| Vitamin B1 | 2-4 | mg/kg feed | 2-3 | mg/kg feed |
| Vitamin B2 | 6-10 | mg/kg feed | 7-9 | mg/kg feed |
| Vitamin B6 | 4-8 | mg/kg feed | 3-6 | mg/kg feed |
| Vitamin B12 | 0.03-0.05 | mg/kg feed | 0.015-0.04 | mg/kg feed |
| Niacin | 30-50 | mg/kg feed | 50-80 | mg/kg feed |
| (Vitamin B3) | | | | |
| Pantothenic | 20-40 | mg/kg feed | 10-18 | mg/kg feed |
| acid | | | | |
| Folic acid | 1-2 | mg/kg feed | 1-2 | mg/kg feed |
| Biotin | 0.15-0.4 | mg/kg feed | 0.15-0.3 | mg/kg feed |
| Choline | 200-400 | mg/kg feed | 300-600 | mg/kg feed |
| chloride | | | | |

### Other Feed Ingredients

the composition, animal feed or animal feed additive of the present invention may further comprise colouring agents, stabilisers, growth improving additives and aroma compounds/flavourings, polyunsaturated fatty acids (PUFAs); reactive oxygen generating species, anti-microbial peptides and anti-fungal polypeptides.

Examples of the colouring agents are carotenoids such as beta-carotene, astaxanthin, and lutein.

Examples of the stabilizing agents (e.g. acidifiers) are organic acids. Examples of these are benzoic acid (VevoVitall®, DSM Nutritional Products), formic acid, butyric acid, fumaric acid and propionic acid.

Examples of the aroma compounds/flavourings are creosol, anethol, deca-, undeca- and/or dodeca-lactones, ionones, irone, gingerol, piperidine, propylidene phatalide, butylidene phatalide, capsaicin and tannin.

Examples of the polyunsaturated fatty acids are C18, C20 and C22 polyunsaturated fatty acids, such as arachidonic acid, docosohexaenoic acid, eicosapentaenoic acid and gamma-linoleic acid.

Examples of the reactive oxygen generating species are chemicals such as perborate, persulphate, or percarbonate; and enzymes such as an oxidase, an oxygenase or a syntethase.

Examples of the antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of the antifungal polypeptides (AFP's) are the Aspergillus giganteus, and Aspergillus niger peptides, as well as variants and fragments thereof which retain antifungal activity, as disclosed in WO 94/01459 and WO 02/090384.

### Use of Microbial Lyzozyme

In another aspect, the invention relates to the use of a composition, an animal feed or an animal feed additive for improving flock uniformity and/or meat quality of a monogastric animal wherein the composition, the animal feed or the animal feed additive comprises one or more microbial muramidases.

In the present invention, the microbial muramidase may be dosed at a level of 100 to 1000 mg enzyme protein per kg animal feed, such as 200 to 900 mg, 300 to 800 mg, 400 to 700 mg, 500 to 600 mg enzyme protein per kg animal feed, or any combination of these intervals.

In the present invention, the monogastric animal may be selected from the group consisting of swine, piglet, growing pig, sow, poultry, turkey, duck, quail, guinea fowl, goose, pigeon, squab, chicken, broiler, layer, pullet and chick, cat, dog, horse, crustaceans, shrimps, prawns, fish, amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish. Preferably, the monogastric animal is a selected from the group consisting of swine, piglet, growing pig, sow, poultry, turkey, duck, quail, guinea fowl, goose, pigeon, squab, chicken, broiler, layer, pullet and chick. More preferably, the the monogastric animal is a selected from the group consisting of swine, piglet, growing pig, sow, chicken, broiler, layer, and chick.

In the present invention, the microbial muramidase may be fed to the animal from birth until slaughter. Preferably, the the microbial muramidase is fed to the animal on a daily basis from birth until slaughter. More Preferably, the microbial muramidase is fed to the animal on a daily basis for at least 10 days, such as at least 15 days or at least 20 days (where the days can be continuous or non-continuous) during the life span of the animal. In one embodiment, the microbial muramidase is fed to the animal for 10-20 days followed by a non-treatment period of 5-10 days, and this cycle is repeated during the life span of the animal.

In the present invention, the microbial muramidase may be fed to broilers for the first 49 days after hatching. Preferably, the microbial muramidase is fed to broilers for the first 36 days after hatching. More preferably, the microbial muramidase is fed to broilers on days 22 to 36 after hatching. Further preferably, the microbial muramidase is fed to broilers during the pre-starter (days 1-7) period. Further preferably, the microbial muramidase is fed to broilers during the starter (days 8-22) period. Further preferably, the microbial muramidase is fed to broilers during the pre-starter (days 1-7) and starter (days 8-22) period.

In the present invention, the microbial muramidase may be fed to layers during the life span of the animal. Preferably, the microbial muramidase is fed to layers for 76 weeks from hatching. More preferably, the microbial muramidase is fed to layers during the laying period, (from ca. week 18). Further preferably, the microbial muramidase is fed to layers during the laying period but withheld during the forced molting period.

In the present invention, the microbial muramidase may be fed to turkeys during life span of the animal. Preferably, the microbial muramidase is fed to turkeys for 24 weeks from hatching. More preferably, the microbial muramidase is fed to turkeys for the first 16 weeks from hatching (for hens) and for the first 20 weeks for hatching (for toms).

In the present invention, the microbial muramidase may be fed to swine during life span of the animal. Preferably, the microbial muramidase is fed to swine for 27 weeks from birth. More preferably, the microbial muramidase is fed to piglets from birth to weaning (at 4 weeks). Further preferably, the microbial muramidase is fed to piglets for the first 6 weeks from birth (4 weeks of lactation and 2 weeks post-weaning). Further preferably, the microbial muramidase is fed to weaning piglets during the pre-starter (days 1-14 after weaning). Further preferably, the microbial muramidase is fed to weaning piglets during the starter (days 15-42 after weaning) period. Further preferably, the microbial muramidase is fed to weaning piglets during the pre-starter (days 1-14 after weaning) and starter (days 15-42 after weaning) period. Further preferably, the microbial muramidase is fed to swine during the grower/fattening period (week 10 to ca. week 27 after birth).

In the present invention, the microbial muramidase may be of fungal origin. Preferably, the microbial muramidase is obtained or obtainable from the phylum Ascomycota, such as the sub-phylum Pezizomycotina. Preferably, the microbial muramidase comprises one or more domains selected from the list consisting of GH24 and GH25.

### EXAMPLES

### Strains

Trichophaea saccata CBS804.70 was purchased from the Centraalbureau voor Schimmelcultures (Utrecht, the Netherlands). According to Central Bureau vor Schnimmelkulture, Trichophaea saccata CBS804.70 was isolated from coal spoil tip soil from Staffordshire, England in May 1968.

According to Central Bureau vor Schnimmelkulture, Acremonium alcalophilum CBS 114.92 was isolated by A. Yoneda in 1984 from the sludge of pig faeces compost near Tsukui Lake, Japan.

### Media and Solutions

YP+2% glucose medium was composed of 1% yeast extract, 2% peptone and 2% glucose.

YP+2% maltodextrin medium was composed of 1% yeast extract, 2% peptone and 2% maltodextrin.

PDA agar plates were composed of potato infusion (potato infusion was made by boiling 300 g of sliced (washed but unpeeled) potatoes in water for 30 minutes and then decanting or straining the broth through cheesecloth). Distilled water was then added until the total volume of the suspension was one liter, followed by 20 g of dextrose and 20 g of agar powder. The medium was sterilized by autoclaving at 15 psi for 15 minutes (Bacteriological Analytical Manual, 8th Edition, Revision A, 1998).

LB plates were composed of 10 g of Bacto-Tryptone, 5 g of yeast extract, 10 g of sodium chloride, 15 g of Bacto-agar, and deionized water to 1 liter.

LB medium was composed of 10 g of Bacto-Tryptone, 5 g of yeast extract, 10 g of sodium chloride, and deionized water to 1 liter.

COVE sucrose plates were composed of 342 g of sucrose, 20 g of agar powder, 20 ml of COVE salts solution, and deionized water to 1 liter. The medium was sterilized by autoclaving at 15 psi for 15 minutes (Bacteriological Analytical Manual, 8th Edition, Revision A, 1998). The medium was cooled to 60° C. and 10 mM acetamide, 15 mM CsCl, TRITON® X-100 (50 μl/500 ml) were added.

COVE salts solution was composed of 26 g of MgSO4.7H2O, 26 g of KCL, 26 g of KH2PO4, 50 ml of COVE trace metals solution, and deionized water to 1 liter.

COVE trace metals solution was composed of 0.04 g of Na2B407.10H2O, 0.4 g of CuSO4.5H2O, 1.2 g of FeSO4.7H2O, 0.7 g of MnSO4.H2O, 0.8 g of Na2MoO4.2H2O, 10 g of ZnSO4.7H2O, and deionized water to 1 liter.

### Example 1: Cloning, Expression and Purification of the GH25 Muramidase from Acremonium alcalophilum CBS 114.92

The GH25 muramidase from Acremonium alcalophilum CBS 114.92 (SEQ ID NO: 1) was cloned and expressed as described in example 8 and purified as described in example 5 of WO 2013/076253. Alternatively, SEQ ID NO: 10 can be cloned and expressed as described in example 2 of WO 2013/076253.

### Example 2: Expression of the GH24 Muramidase from Trichophaea saccata

The fungal strain was cultivated in 100 ml of YP+2% glucose medium in 1000 ml Erlenmeyer shake flasks for 5 days at 20° C. Mycelia were harvested from the flasks by filtration of the medium through a Buchner vacuum funnel lined with MIRACLOTH® (EMD Millipore, Billerica, Mass., USA). Mycelia were frozen in liquid nitrogen and stored at −80° C. until further use. Genomic DNA was isolated using a DNEASY® Plant Maxi Kit (QIAGEN GMBH, Hilden Germany) according to the manufacturer's instructions.

Genomic sequence information was generated by Illumina MySeq (Illumina Inc., San Diego, Calif.). 5 μgs of the isolated Trichophaea saccata genomic DNA was used for library preparation and analysis according to the manufacturer's instructions. A 100 bp, paired end strategy was employed with a library insert size of 200-500 bp. One half of a HiSeq run was used for the total of 95,744,298, 100 bp raw reads obtained. The reads were subsequently fractionated to 25% followed by trimming (extracting longest sub-sequences having Phred-scores of 10 or more). These reads were assembled using Idba version 0.19. Contigs shorter than 400 bp were discarded, resulting in 8,954,791,030 bp with an N-50 of 10,035. Genes were called using GeneMark.hmm ES version 2.3c and identification of the catalytic domain was made using “Phage muramidase PF00959” Hidden Markov Model provided by Pfam. The polypeptide coding sequence for the entire coding region was cloned from Trichophaea saccata CBS804.70 genomic DNA by PCR using the primers F-80470 and R-80470 (SEQ ID NO: 6 and SEQ ID NO: 7 respectively) as described below.

(SEQ ID NO: 6)
5′-ACACAACTGGGGATCCACCATGCACGCTCTCACCCTTCT-3′
(SEQ ID NO: 7)
5′-CTAGATCTCGAGAAGCTTTTAGCACTTGGGAGGGTGGG-3′

Bold letters represent Trichophaea saccata enzyme coding sequence. Restriction sites are underlined. The sequence to the left of the restriction sites is homologous to the insertion sites of pDau109 (WO 2005/042735).

Extensor HIFI PCR mix, 2× concentration (Thermo Scientific cat no AB-0795) was used for experiment.

The amplification reaction (25 μl) was performed according to the manufacturer's instructions (Thermo Scientific cat no AB-0795) with the following final concentrations:

PCR mix:

0.5 μM Primer F-80470

0.5 μM Primer R-80470

12.5 μl Extensor HIFI PCR mix, 2× conc.

11.0 μl H2O

10 ng of Trichophaea saccata CBS804.70 genomic DNA.

The PCR reaction was incubated in a DYAD® Dual-Block Thermal Cycler (BioRad, USA) programmed for 1 cycle at 94° C. for 30 seconds; 30 cycles each at 94° C. for 30 seconds, 52° C. for 30 seconds and 68° C. for 60 seconds followed by 1 cycle at 68° C. for 6 minutes. Samples were cooled to 10° C. before removal and further processing.

Three μl of the PCR reaction were analyzed by 1% agarose gel electrophoresis using 40 mM Tris base, 20 mM sodium acetate, 1 mM disodium EDTA (TAE) buffer. A major band of about 946 bp was observed. The remaining PCR reaction was purified directly with an ILLUSTRA™ GFX™ PCR DNA and Gel Band Purification Kit (GE Healthcare, Piscataway, N.J., USA) according to the manufacturer's instructions.

Two μg of plasmid pDau109 was digested with Bam HI and Hind III and the digested plasmid was run on a 1% agarose gel using 50 mM Tris base-50 mM boric acid-1 mM disodium EDTA (TBE) buffer in order to remove the stuffer fragment from the restricted plasmid. The bands were visualized by the addition of SYBR® Safe DNA gel stain (Life Technologies Corporation, Grand Island, N.Y., USA) and use of a 470 nm wavelength transilluminator. The band corresponding to the restricted plasmid was excised and purified using an ILLUSTRA™ GFX™ PCR DNA and Gel Band Purification Kit. The plasmid was eluted into 10 mM Tris pH 8.0 and its concentration adjusted to 20 ng per μl. An IN-FUSION® PCR Cloning Kit (Clontech Laboratories, Inc., Mountain View, Calif., USA) was used to clone the 983 bp PCR fragment into pDau109 digested with Bam HI and Hind III (20 ng). The IN-FUSION® total reaction volume was 10 μl. The IN-FUSION® total reaction volume was 10 μl. The IN-FUSION® reaction was transformed into FUSION-BLUE™ E. coli cells (Clontech Laboratories, Inc., Mountain View, Calif., USA) according to the manufacturer's protocol and plated onto LB agar plates supplemented with 50 μg of ampicillin per ml. After incubation overnight at 37° C., transformant colonies were observed growing under selection on the LB plates supplemented with 50 μg of ampicillin per ml.

Several colonies were selected for analysis by colony PCR using the pDau109 vector primers described below. Four colonies were transferred from the LB plates supplemented with 50 μg of ampicillin per ml with a yellow inoculation pin (Nunc NS, Denmark) to new LB plates supplemented with 50 μg of ampicillin per ml and incubated overnight at 37° C.

Primer 8653:
(SEQ ID NO: 8)
5′-GCAAGGGATGCCATGCTTGG-3′
Primer 8654:
(SEQ ID NO: 9)
5′-CATATAACCAATTGCCCTC-3′

Each of the three colonies were transferred directly into 200 μl PCR tubes composed of 5 μl of 2× Extensor HIFI PCR mix, (Thermo Fisher Scientific, Rockford, Ill., USA), 0.5 μl of primer 8653 (10 pm/μl), 0.5 μl of primer 8654 (10 pm/μl), and 4 μl of deionized water. Each colony PCR was incubated in a DYAD® Dual-Block Thermal Cycler programmed for 1 cycle at 94° C. for 60 seconds; 30 cycles each at 95° C. for 30 seconds, 60° C. for 45 seconds, 72° C. for 60 seconds, 68° C. for 10 minutes, and 10° C. for 10 minutes.

Three μl of each completed PCR reaction were submitted to 1% agarose gel electrophoresis using TAE buffer. All four E. coli transformants showed a PCR band of about 980 bp. Plasmid DNA was isolated from each of the four colonies using a QIAprep Spin Miniprep Kit (QIAGEN GMBH, Hilden Germany). The resulting plasmid DNA was sequenced with primers 8653 and 8654 (SEQ ID NO: 8 and 9) using an Applied Biosystems Model 3730 Automated DNA Sequencer using version 3.1 BIG-DYE™ terminator chemistry (Applied Biosystems, Inc., Foster City, Calif., USA). One plasmid, designated pKKSC0312-2, was chosen for transforming Aspergillus oryzae MT3568. A. oryzae MT3568 is an amdS (acetamidase) disrupted gene derivative of Aspergillus oryzae JaL355 (WO 2002/40694) in which pyrG auxotrophy was restored by inactivating the A. oryzae amdS gene. Protoplasts of A. oryzae MT3568 were prepared according to the method described in European Patent, EP0238023, pages 14-15.

E. coli 3701 containing pKKSC0312-2 was grown overnight according to the manufacturer's instructions (Genomed) and plasmid DNA of pKKSC0312-2 was isolated using a Plasmid Midi Kit (Genomed JETquick kit, cat.nr. 400250, GENOMED GmbH, Germany) according to the manufacturer's instructions. The purified plasmid DNA was transformed into Aspergillus oryzae MT3568. A. oryzae MT3568 protoplasts were prepared according to the method of Christensen et al., 1988, Bio/Technology 6: 1419-1422. The selection plates consisted of COVE sucrose with +10 mM acetamide+15 mM CsCl+TRITON® X-100 (50 μl/500 ml). The plates were incubated at 37° C. Briefly, 8 μl of plasmid DNA representing 3 ugs of DNA was added to 100 μl MT3568 protoplasts. 250 μl of 60% PEG solution was added and the tubes were gently mixed and incubate at 37° for 30 minutes. The mix was added to 10 ml of pre melted Cove top agarose (The top agarose melted and then the temperature equilibrated to 40 C in a warm water bath before being added to the protoplast mixture). The combined mixture was then plated on two Cove-sucrose selection petri plates with 10 mM Acetamide. The plates were incubated at 37° C. for 4 days. Single Aspergillus transformed colonies were identified by growth on plates using the selection Acetimide as a carbon source. Each of the four A. oryzae transformants were inoculated into 750 μl of YP medium supplemented with 2% glucose and also 750 μl of 2% maltodextrin and also DAP4C in 96 well deep plates and incubated at 37° C. stationary for 4 days. At the same time the four transformants were restreaked on COVE-2 sucrose agar medium.

Culture broth from the Aspergillus oryzae transformants were then analyzed for production of the GH24 polypeptide by SDS-PAGE using NUPAGE® 10% Bis-Tris SDS gels (Invitrogen, Carlsbad, Calif., USA) according to the manufacturer's recommendations. A protein band at approximately 27 kDa was observed for each of the Aspergillus oryzae transformants. One A. oryzae transformant was cultivated in 1000 ml Erlenmeyer shake flasks containing 100 ml of DAP4C medium at 26° C. for 4 days with agitation at 85 rpm.

### Example 3: Purification of the GH24 Muramidase from Trichophaea saccata

The fermentation supernatant with the GH24 muramidase from example 2 was filtered through a Fast PES Bottle top filter with a 0.22 μm cut-off. The resulting solution was diafiltrated with 5 mM Na-acetate, pH 4.5 and concentrated (volume reduced by a factor of 10) on an Ultra Filtration Unit (Sartorius) with a 10 kDa cut-off membrane.

After pretreatment about 275 mL of the muramidase containing solution was purified by chromatography on SP Sepharose (approximately 60 mL) in a XK26 column eluting the bound muramidase with 0 to 100% gradient of buffer A (50 mM Na-acetate pH 4.5) and buffer B (50 mM Na-acetate+1 M NaCl pH 4.5) over 10 column volumes. The fractions from the column were pooled based on the chromatogram (absorption at 280 and 254 nm) and SDS-PAGE analysis.

The molecular weight, as estimated from SDS-PAGE, was approximately 27 kDa and the purity was >90%.

### Example 4: Other Characteristics for the GH24 Muramidase from Trichophaea saccata

Determination of the N-terminal sequence was: YPVKTDL.
The calculated molecular weight from this mature sequence is 26205.5 Da (M+H)⁺.
The molecular weight determined by intact molecular weight analysis was 26205.3 Da. (M+H)⁺.
The mature sequence (from EDMAN N-terminal sequencing data, intact molecular weight analysis and proteomic analysis):

(SEQ ID NO: 4)
YPVKTDLHCRSSPSTSASIVRTYSSGTEVQIQCQTTGTSVQGSNVWDKT
QHGCYVADYYVKTGHSGIFTTKCGSSSGGGSCKPPPINAATVALIKEFE
GFVPKPAPDPIGLPTVGYGHLCKTKGCKEVPYSFPLTQETATKLLQSDI
KTFTSCVSNYVKDSVKLNDNQYGALASWAFNVGCGNVQTSSLIKRLNAG
ENPNTVAAQELPKWKYAGGKVMPGLVRRRNAEVALFKKPSSVQAHPPK
C.

### Example 5: Determination of Muramidase Activity

Muramidase activity was determined by measuring the decrease (drop) in absorbance/optical density of a solution of resuspended Micrococcus lysodeikticus ATTC No. 4698 (Sigma-Aldrich M3770) or Exiguobacterium undea (DSM14481) measured in a spectrophotometer at 540 nm.

### Preparation of Micrococcus Lysodeikticus Substrate

Before use the cells were resuspended in citric acid—phosphate buffer pH 6.5 to a concentration of 0.5 mg cells/mL and the optical density (OD) at 540 nm was measured. The cell suspension was then adjusted so that the cell concentration equalled an OD540=1.0. The adjusted cell suspension was then stored cold before use. Resuspended cells were used within 4 hours.

Preparation of Dried Cells of Exiguobacterium undae Substrate

A culture of E. undae (DSM14481) was grown in 100 mL LB medium (Fluka 51208, 25 g/L) in a 500 mL shake-flask at 30° C., 250 rpm overnight. The overnight culture was then centrifuged at 20° C. and 5000 g for 10 minutes, and the pellet was then washed twice with sterile milliQ water, and resuspended in Milli-Q water. The washed cells were centrifuged for 1 minute at 13000 rpm and as much as possible of the supernatant was decanted. The washed cells were dried in a vacuum centrifuge for 1 hour. The cell pellet was resuspended in citric acid—phosphate buffer pH 4, 5 or 6 so that the optical density (OD) at 540 nm=1.

### Measurement of Muramidase Antimicrobial Activity in the Turbidity Assay

The muramidase sample to be measured was diluted to a concentration of 100-200 mg enzyme protein/L in citric acid—phosphate buffer pH 4, 5 or 6, and kept on ice until use. In a 96 well microtiterplate (Nunc) 200 μL of the substrate was added to each well, and the plate was incubated at 37° C. for 5 minutes in a VERSAmax microplate reader (Molecular Devices). Following incubation, the absorbance of each well was measured at 540 nm (start value). To start the activity measurement, 20 μL of the diluted muramidase sample was added to each substrate (200 μL) and kinetic measurement of absorbance at 540 nm was initiated for minimum 30 minutes up to 24 hours at 37° C. The measured absorbance at 540 nm was monitored for each well and over time a drop in absorbance is seen if the muramidase has muramidase activity. The results are presented in table 2 below.

TABLE 2
Muramidase Activity against Micrococcus lysodeikticus and
Exiguobacterium undea as measured by Optical Density Drop

| | Micrococcus | Exiguobacterium |
|---|---|---|
| Muramidase | lysodeikticus¹ | undae¹ |
| GH22 muramidase from Gallus | +++ (pH 6) | + (pH 6) |
| gallus | | |
| (SEQ ID NO: 5) | | |
| GH24 muramidase from | ++ (pH 6) | ++ (pH 6) |
| Trichophaea saccata (SEQ ID NO: | | |
| 4) | | |
| GH25 muramidase from A. | + (pH 4) | + (pH 5) |
| alcalophilum | | |
| (SEQ ID NO: 1) | | |

¹Means no effect; + means small effect; ++ means medium effect; +++ means large effect. The pH value in the brackets lists the assay pH based on muramidase-substrate combination.

The data confirms that the GH22 muramidase from Gallus gallus, the GH24 muramidase from Trichophaea saccata and the GH25 muramidase from A. alcalophilum all have muramidase activity.

### Example 6: In Vivo Broiler Trial 1

### Materials and Methods

Trial (ME-21/16) was performed from Aug. 30 to Oct. 5, 2016 at the Research Center for Animal Nutrition (DSM Nutritional Products France, F-68305 Village-Neuf) according to the official French guidelines for experiments with live animals.

### Animals and Housing

Day-old male broiler chickens (Cobb 500) were supplied by a commercial hatchery (Joseph Grelier S. A., Elevage avicole de la Bohadiére, F-49290 Saint-Laurent de la Plaine, France).

On the day of arrival (day 1), the chickens were divided by weight into groups of 18 birds. Each group was placed in one floor-pen littered with wood shavings and allocated to one of the different treatments. Each treatment was replicated with 8 groups. Chickens were housed in an environmentally controlled room. The room temperature was adapted to the age of the birds. In the first few days an additional infra-red electric heating lamp was placed in each pen. Moreover, in the first week, feed was offered to the birds as crumbled pellets, afterwards as pelleted feed. Birds had free access to feed and water.

### Feeding and Treatments

The experimental diets (Starter and Grower) were based on soybean meal, corn, wheat and rye as main ingredients (Table 3). The diets were formulated to contain 211 g/kg crude protein and 12.4 MJ/kg ME for the starter period and 191 g/kg crude protein and 12.7 MJ/kg ME for the grower period. Ronozyme HiPhos at 100 mg/kg, Ronozyme ProAct at 200 mg/kg and Carophyl yellow 60 mg/kg (10% ApoEster) were included in all the basal diets.

TABLE 3
Composition and nutrient contents of the basal experimental diets

| | Starter (d 1-22) | Grower (d 22-36) |
|---|---|---|
| Ingredients | Inclusion (%) | Inclusion (%) |
| Wheat | 45.00 | 45.00 |
| Rye | 10.00 | 10.00 |
| SBM | 32.00 | 26.60 |
| Maize | 6.48 | 10.80 |
| Vegetable Oil | 3.00 | 3.80 |
| NaCl | 0.20 | 0.20 |
| DL Methionine | 0.22 | 0.22 |
| L-Lysine | 0.20 | 0.22 |
| Limestone | 0.90 | 0.86 |
| Dical Phos | 1.00 | 1.30 |
| V&M | 1.00 | 1.00 |
| TiO₂ | — | 0.10 |
| Calculated Provision | | |
| AME, MJ/kg | 12.4 | 12.7 |
| AME, Kcal/kg | 2963 | 3034 |
| Crude Protein, % | 21.1 | 19.1 |
| Met + Cys, % | 0.87 | 0.82 |
| Lys, % | 1.23 | 1.11 |
| Ca, % | 0.70 | 0.75 |
| P total, % | 0.55 | 0.58 |
| avP, % | 0.28 | 0.32 |
| Analyzed content | | |
| Crude protein (%) | 209 | 183 |

Inclusion of Ronozyrne HiPhos at 100 mg/kg, Ronozyme ProAct at 200 mg/kg and Carophyl yellow at 60 mg/kg (10% Apo-ester) on top in the basal diet
¹ Vitamin-mineral premix provided per kilogram of diet: Vitamin A: 10'000 I.U.; vitamin E: 40 I.U.; vitamin K3: 3.0 mg; vitamin C: 100 mg; vitamin B1: 2.50 mg; vitamin B2: 8.00 mg; vitamin B6: 5.00 mg; vitamin B12: 0.03 mg; niacin: 50.0 mg; pantothenate calcium: 12.0 mg; folic acid: 1.50 mg; biotin 0.15 mg; cholin: 450 mg; ethoxyquine: 54 mg; Na: 1.17 g; Mg: 0.8 g; Mn: 80 mg; Fe: 60 mg; Cu: 30 mg; Zn: 54 mg; I: 1.24 mg; Co: 0.6 mg; Se: 0.3 mg
² Metabolizable Energy calculated with EC-equation based on analyzed crude nutrients according to the formula ME (MJ/kg) = ((15.51*crude protein + 34.31*fat + 16.69*starch + 13.01*sugar)/1000)

Diets were fed either non-supplemented (negative control) or supplemented with the following products:

TABLE 4

| | | Dosage |
|---|---|---|
| Group name | Description | (LSU/kg feed) |
| A | Negative Control (NC) | — |
| B | Muramidase Low | 25 000 LSU/kg |
| C | Muramidase High | 35 000 LSU/kg |

### Experimental Parameters and Analyses

All broilers were weighed individually at 1, 22 and 36 days of age to estimate the coefficient of variation (CV). The CV is a measure of relative variability, and it is calculated as the ratio of the standard deviation to the mean (average) and reported as percentage of coefficient of variability.

% CV=(standard deviation/mean×100).

Percentage of CV is an essential indicator of the productive process, high percentage is correlated with poor uniformity and performances. Chick flocks with lower uniformity cannot be properly managed and plays an important issue for the processing plant. This situation, results in lower growth, increased feed intake, and mortality during the first weeks. Based on studies with several broiler flocks, the CV of individual BW from a single flock may be between 7.5 to 10.7%, with an average of approximately 9.25%. In addition, broiler flocks with higher values of the CV (reduced uniformity) experienced higher mortality rates. This measure of dispersion can indicate the zootechnical quality of flocks; lower CV corresponds to higher chick quality (Shalev et, al., 1995).

### Results and Discussion

As shown in Table 5, the flock uniformity in the present study varied between 3.10%, in the treatment including Muramidase at 35 000 LSU(F)/kg to 5.69%, in the NC treatment, at the end of the study (Table 5).

TABLE 5
Flock uniformity.

| Treatment/Product | WG D1-22 | WG D22-36 | WG D1-36 |
|---|---|---|---|
| A NC | 3.36 | 10.30 | 5.69 |
| B Muramidase Low | 3.26 | 6.06 | 3.72 |
| C Muramidase High | 3.08 | 6.85 | 3.10 |

### Conclusion

The results obtained in the study showed that the inclusion of microbial muramidase was effective in improving flock uniformity of broilder chickens.

### Example 7: In Vivo Broiler Trial 2

### Materials and Methods

The trial was performed at Universidade Federal do Parana in Parana, Brazil from July 2017 to Febrary 2018.

### Animal and Housing

A total amount of 1000 day-old-chicks (without antibiotics at hatchery), males, Cobb 500, were placed in floor pen boxes in a completely randomized design. There were 4 treatments, 10 replicates per treatment with 25 birds each. Broilers were raised in a reused litter for 42 days. Chickens housed in floor pens, distributed in 110 boxes of 2.06 m².

The treatments were established as follow:

TABLE 7

| Group name | Description | Dosage (LSU/kg feed) |
|---|---|---|
| T1 | Negative Control (NC) | — |
| T2 | Muramidase Low | 25 000 LSU/kg (392 mg/kg) |

### Experimental Diets

In this trial were used vegetable mash diets, based on corn and SBM, manufactured at research UFPR station. The experimental diets will be formulated in accordance to components as shown in Table 8 for 3 phases: starter (d1-d21), grower (d21-d35) and finisher (d35-d42). Additionally, in all treatments were added: phytase (RONOZYME® HiPhos GT) 1000 FYT/kg (100 ppm), xylanase (RONOZYME WX 2000 CT) 100 FXU/kg (50 ppm) and 40 mg/kg of CAROPHYLL® yellow 10%.

TABLE 8
composition of experimental Diets

| | Starter | Grower | Finisher |
|---|---|---|---|
| | 1-21 days | 22-35 days | 36-42 days |
| Ingredients, % | | | |
| CORN | 553,130 | 606,860 | 606,140 |
| SOYBEAN MEAL | 388,000 | 336,000 | 314,000 |
| SALT | 4,800 | 4,300 | 4,100 |
| CHOLINE CHLORIDE | 0,800 | 0,600 | 0,500 |
| SOYBEAN OIL | 24,000 | 28,000 | 45,000 |
| DICALCIUM PHOSPHATE | 10,000 | 6,800 | 4,500 |
| LIMESTONE | 12,000 | 10,800 | 9,900 |
| DL-METHIONINE | 2,970 | 2,670 | 2,480 |
| L-LYSINE | 0,910 | 0,590 | 0,030 |
| L-THREONINE | 0,100 | 0,090 | 0,140 |
| PX ROVIMIX AVES (DSM)* | 1,500 | 1,500 | 1,000 |
| PX ROLIGOMIX AVES (DSM)** | 0,500 | 0,500 | 0,500 |
| HiPhos GT | 0,100 | 0,100 | 0,100 |
| RONOZYME WX 2000 | 0,050 | 0,050 | 0,050 |
| BHT | 0,100 | 0,100 | 0,100 |
| CAROPHYLL ® yellow 10% | 0,040 | 0,040 | 0,000 |
| Celite ® | 0,00 | 0,00 | 10,000 |
| Vehicle | 1,000 | 1,000 | 1,440 |
| | 1.000,000 | 1.000,000 | 1.000,000 |
| Calculated analysis, % | | | |
| AMEn, kcal/kg | 3.000,00 | 3.100,00 | 3.200,00 |
| Crude Protein | 22,00 | 20,00 | 19,00 |
| Calcium | 0,94 | 0,80 | 0,70 |
| Av. P | 0,47 | 0,40 | 0,35 |
| Lys dig | 1,20 | 1,05 | 0,95 |
| Met dig | 0,60 | 0,55 | 0,52 |
| Thr dig | 0,78 | 0,71 | 0,68 |
| Trp dig | 0,26 | 0,23 | 0,22 |
| Arg dig | 1,42 | 1,27 | 1,20 |

*PX ROVIMIX AVES (DSM): Vit. A 9,000,000 UI/kg; Vit. D3 2,500,000 UI/kg; Vit. E 20,000 UI/kg; Vit. K3 2,500 mg/kg; Vit. B1 2,000 mg/kg; Vit. B2 6,000 mg/kg; Pantotenic acid 12 g/kg; Vit. B6 3,000 mg/kg; Vit. B12 15,000 mcg/kg; Nicotinic acid 35 g/kg; Folic acid 1,500 mg/kg; Biotin 100 mg/kg; Selenium 250 mg per kg of premix.
**PX ROLIGOMIX AVES (DSM): Iron 100 g/kg; Cooper 20 g/kg; Manganese 130 g/kg; Cobalt 2,000 mg/kg; Zinc 130 mg/kg; Iodine 2,000 mg per kg of premix.

Experimental Measurements and Procedures

Meat Quality: Carcass, breast, and breast fillet meat yield were weighted at 42 days of age, 10 birds/replicate from T1 and T2, total 100 birds/treatment.

### Results and Discussion

Carcass yield data, at 42 days of age, are shown in Table 6 and 7, were percentages of carcass, breast and breast filet calculated in relation to live body weight and carcass weight respectively.

TABLE 6
Broiler chicken carcass,
breast, and breast filet yield (%) with 42 days of age.

| Treatments | Carcass | Breast* | Breast Filet* |
|---|---|---|---|
| Negative Control | 80.426 | 31.506 ^{b} | 19.626 ^{b} |
| 25,000 LSU (F)/kg | 80.539 | 33.310 ^{a} | 20.881 ^{a} |
| CV (%) | 1.95 | 8.67 | 9.44 |
| P-value | 0.623 | <.0001 | <.0001 |

*Calculated based on live body weight.

TABLE 7
Broiler chicken carcass, breast, and breast filet yield (%) with 42 days of age.

| Treatments | Breast* | Breast Filet* |
|---|---|---|
| Negative Control | 38.42 ^{b} | 23.80 |
| 25,000 LSU (F)/kg | 39.05 ^{a} | 24.08 |
| CV (%) | 5.06 | 6.74 |
| P-value | 0.032 | 0.251 |

*Calculated based on carcass weight.

As shown above, broilers that received muramidase in the diet had a higher percentage of breast and breast filet compare to control.

### Conclusions

The results obtained in the study showed that the inclusion of microbial muramidase was effective in improving meat quality of broiler chickens.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A method of improving flock uniformity and/or meat quality of a monogastric animal comprising administering to the animal a composition, an animal feed or an animal feed additive comprising one or more microbial muramidases.

2. The method of claim 1, wherein the monogastric animal is selected from the group consisting of swine, piglet, growing pig, sow, poultry, turkey, duck, quail, guinea fowl, goose, pigeon, squab, chicken, broiler, layer, pullet and chick, cat, dog, horse, crustaceans, shrimps, prawns, fish, amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish.

3. The method of claim 2, wherein the monogastric animal is selected from the group consisting of swine, piglet, growing pig, sow, chicken, broiler, layer, pullet and chick.

4. The method of claim 1, wherein the microbial muramidase is obtained or obtainable from the phylum Ascomycota, or the subphylum Pezizomycotina.

5. The method of claim 1, wherein the microbial muramidase comprises one or more domains selected from the list consisting of GH24 and GH25.

6. The method of claim 1, wherein the microbial muramidase is selected from the group consisting of:
(a) a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
(b) a variant of SEQ ID NO: 1 wherein the variant has muramidase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(c) a fragment of the polypeptide of (a) or (b) that has muramidase activity wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids;
(d) a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
(e) a variant of SEQ ID NO: 4 wherein the variant has muramidase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; and
(f) a fragment of the polypeptide of (d) or (e) that has muramidase activity wherein the fragment comprises at least 210 amino acids, such as at least 215 amino acids, at least 220 amino acids, at least 225 amino acids, at least 230 amino acids, at least 235 amino acids or at least 240 amino acids.

7. The method of claim 1, wherein the microbial muramidase is selected from the group consisting of amino acids 1 to 213 of SEQ ID NO: 1, amino acids 1 to 245 of SEQ ID NO: 4 and amino acids 1 to 208 of SEQ ID NO: 10.

8. The method of claim 1, wherein the composition, the animal feed or the animal feed additive further comprises one or more components selected from the list consisting of:
one or more carriers;
one or more additional enzymes;
one or more microbes;
one or more vitamins;
one or more minerals;
one or more amino acids;
one of more organic acids; and
one or more other feed ingredients.

9. A method of improving flock uniformity and/or meat quality of a monogastric animal comprising administering to the animal a composition, an animal feed or an animal feed additive comprising one or more microbial muramidases, wherein:
(a) the microbial muramidase is a microbial muramidase comprising one or more domains selected from the list consisting of GH24 and GH25, is dosed at a level of 300 to 500 mg enzyme protein per kg animal feed;
(b) the monogastric animal is a selected from the group consisting of swine, piglet, growing pig, sow, chicken, broiler, layer, pullet and chick;
(c) optionally the microbial muramidase is fed to the animal on a daily basis for at least 10 days during the life span of the animal.

10. A method of improving flock uniformity and/or meat quality of a monogastric animal comprising administering to the animal a composition, an animal feed or an animal feed additive comprising one or more microbial muramidases, wherein:
(a) the microbial muramidase is a GH24 or GH25 muramidase obtained or obtainable from the phylum Ascomycota, and is dosed at a level of 300 to 500 mg enzyme protein per kg animal feed;
(b) the monogastric animal is a selected from the group consisting of swine, piglet, growing pig, sow, chicken, broiler, layer, pullet and chick; and
(c) the footpad dermatitis is reduced by at least 1% compared to the negative control.

11. Use of a composition, an animal feed or an animal feed additive for improving flock uniformity and/or meat quality of a monogastric animal wherein the composition, the animal feed or the animal feed additive comprises one or more microbial muramidases.

12. The use of claim 11, wherein the monogastric animal is selected from the group consisting of swine, piglet, growing pig, sow, poultry, turkey, duck, quail, guinea fowl, goose, pigeon, squab, chicken, broiler, layer, pullet and chick, cat, dog, horse, crustaceans, shrimps, prawns, fish, amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish.

13. The use of claim 12, wherein the monogastric animal is selected from the group consisting of swine, piglet, growing pig, sow, chicken, broiler, layer, pullet and chick.

14. The use of claim 11, wherein the microbial muramidase is obtained or obtainable from the phylum Ascomycota, or the subphylum Pezizomycotina.

15. The use of claim 11, wherein the microbial muramidase comprises one or more domains selected from the list consisting of GH24 and GH25.

16. The use of claim 11, wherein the microbial muramidase is selected from the group consisting of:
(a) a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
(b) a variant of SEQ ID NO: 1 wherein the variant has muramidase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(c) a fragment of the polypeptide of (a) or (b) that has muramidase activity wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids;
(d) a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4;
(e) a variant of SEQ ID NO: 4 wherein the variant has muramidase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions; and
(f) a fragment of the polypeptide of (d) or (e) that has muramidase activity wherein the fragment comprises at least 210 amino acids, such as at least 215 amino acids, at least 220 amino acids, at least 225 amino acids, at least 230 amino acids, at least 235 amino acids or at least 240 amino acids.

17. The use of claim 11, wherein the microbial muramidase is selected from the group consisting of amino acids 1 to 213 of SEQ ID NO: 1, amino acids 1 to 245 of SEQ ID NO: 4 and amino acids 1 to 208 of SEQ ID NO: 10.


SEQUENCE LISTING (10 sequences)

SEQ ID NO: 1 - protein, 213 aa, Artificial Sequence
  Wild type sequence with N-terminal SPIRR
SPIRRRIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 2 - DNA, 946 bp, Trichophaea saccata
  CDS (1)..(347)
  sig_peptide (1)..(51)
  mat_peptide (52)..(943)
  CDS (401)..(615)
  CDS (668)..(772)
  CDS (825)..(943)
atgcacgctctcacccttctcaccgcaaccctcttcggtctcgcagcggcctacccagtgaagaccgaccttcactgccgctcctctcccagcacttccgccagcatcgtccgcacctactccagtggaacggaagtccagatccagtgccagaccacgggcacttcggtccaaggatccaatgtctgggacaagacccagcacggttgctacgtcgcagactactacgtcaagaccgggcattctgggattttcaccaccaagtgcggtagcagctcgggtggaggttcctgcaagcctcccccgatcaatgctgctactgtcgcattgatcaaggagtttgaggggtaagtgacagctctgagtgaggtggtatgaggattaagactgacgaggatagattcgttcctaagcccgccccggatcctattggattgccgaccgtgggatacgggcatctttgcaagactaagggctgcaaagaagtgccttacagcttccctctcacccaggagactgccaccaagttgcttcagagcgatatcaagactttcacctcttgcgttagcaactacgtcaaggactctgttaagctcaacgataaccagtacggaggtgagttccagtgtaacagtgaatttattgatgatattctaagtaattttagctctggcgtcttgggctttcaacgtcggctgcggaaacgtccagacttcttcgctgatcaagagattgaacgctggggagaaccctaacactgtcgctgctcagggtaagatatttatcccggatttgctcttgacacatggctgaaaaagttgcagaactccccaagtggaagtacgctggtggaaaggttatgcctggcttggtccgccgccgcaatgctgaggtcgcgctcttcaagaagcccagcagcgttcaggcccaccctcccaagtgctaa

SEQ ID NO: 3 - protein, 262 aa, Trichophaea saccata
MHALTLLTATLFGLAAAYPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 4 - protein, 245 aa, Trichophaea saccata
  mat_peptide (1)..(245)
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 5 - protein, 129 aa, Gallus gallus [sequence omitted]

SEQ ID NO: 6 - DNA, 39 bp, Artificial Sequence
  Primer F-80470
acacaactggggatccaccatgcacgctctcacccttct

SEQ ID NO: 7 - DNA, 38 bp, Artificial Sequence
  Primer R-80470.
ctagatctcgagaagcttttagcacttgggagggtggg

SEQ ID NO: 8 - DNA, 20 bp, Artificial Sequence
  Primer 8643.
gcaagggatgccatgcttgg

SEQ ID NO: 9 - DNA, 19 bp, Artificial Sequence
  Primer 8654.
catataaccaattgccctc

SEQ ID NO: 10 - protein, 213 aa, Acremonium alcalophilum; identical to SEQ ID NO: 1